(19) United States
(12) Patent Application Publication
(10) Pub. No.: US 2002/0108620 A1
(43) Pub. Date: Aug. 15, 2002

(54) Informed consent system and method therefor

(76) Inventors: Carl Lauryssen, Ladue, MO (US); Patricia Ann Gelnar, Syracuse, NY (US)

Correspondence Address: Caroline G. Chicoine, Tompson Coburn LLP, One Firstar Plaza, St. Louis, MO, 63101, US

(21) Appl. No.: 09/782,128

(22) Filed: Feb. 13, 2001

(51) Int. Cl. A61B 19/00

(52) U.S. Cl. 128/898

(57) ABSTRACT

An improved informed consent system and method is disclosed. The system comprises a visual component, an auditory component, and a textual component. The visual component consists of a visual representation of the surgery, preferably in three-dimensional form. The auditory component consists of a narration integral with the visual component that explains the visual representation of the surgery. The textual component consists of a summary of each complication associated with the surgery. The system includes an input mechanism for inputting an acknowledgment of each complication, as well as each complication encountered by the patient during the surgery, and a storage mechanism for electronically storing the patient's acknowledgment of each complication. The visual, auditory, and textual components are preferably integrally combined to form a single informed consent system.

A model of the invention in the form of a compact disc is attached hereto as Appendix A, and incorporated herein by reference.

### BACKGROUND OF THE INVENTION

The present invention relates to a system and method of obtaining informed consent from a patient, and more particularly, to a three-dimensional informed consent system and method that uses multiple teaching modalities.

According to the American Medical Association (AMA), in 1998 there were 140,236 surgeons, anesthesiologists and cardiologists in the U.S. performing 71.9 million invasive medical procedures per year in the U.S alone. The number of procedures performed was projected to increase at least 5%-10% every 5 years for the next 25 years. Before a patient undergoes surgery, the surgeon must obtain his or her informed consent. The primary method of surgical informed consent is a verbal consent process, which consists of the surgeon explaining verbally to a patient and/or his or her family how a procedure is performed and the risks associated with surgery. The quality and consistency of verbal informed consents vary significantly between surgeons because of variations in communication skills, time available, and a surgeon's ability and willingness to outline all of the possible complications. This is particularly a problem in teaching hospitals (367 such hospitals in the United States alone), as informed consent is usually obtained by inexperienced doctors in training (interns) or resident physicians who often have a poor understanding of the surgery and potential complications, and typically are functioning on very little sleep. These variables result in disparities in the surgical informed consent. Because of this considerable variability, it is quite common for patients to feel that they are not adequately informed about the procedure and all of the risks involved.

One of the most common reasons for medical malpractice suits after surgery is a patient's contention that the physician did not fully explain the risks and potential complications associated with surgery. Given the inadequacies of the current informed consent procedures, any unforeseen or unsatisfactory outcome can easily lead to litigation. These problems are exacerbated in the area of complex, high risk medical procedures, such as neurosurgery, spine surgery, cardiothoracic surgery, vascular surgery, and obstetrics.

Further, medical malpractice cases typically are more likely to go to trial than any other type of civil case, last longer, and consistently result in high monetary awards to plaintiffs. Given the number of medical procedures performed each year, and the projected increase in such numbers, the loss in time and costs for defending such cases is an enormous burden on physicians, hospitals, and ultimately patients. An additional challenge for surgeons in the near future is an increased need for surgery in our aging population. Therefore, in order to meet this increasing need, surgeons need to become more effective and efficient in the manner in which they handle informed consent.

In addition, insurance premiums are based in part on the type and number of complications typically encountered in connection with the surgery. However, it is very difficult to reliably and automatically track the outcome of the surgery (i.e., the type and number of complications encountered) on a large scale basis. Moreover, there are no existing ways to automatically and reliably track outcomes on a doctor-by-doctor basis.

Several informed consent systems have been developed to try to overcome some of these problems. Some systems employ written materials to help educate patients. Such systems, however, require the physicians to be present during review of the material, and ultimately still rely mainly on a subsequent verbal explanation of the surgery. Other systems employ videotapes. Such tapes, however, use simple animations, which cannot convey accurate surgical relationships and information. In addition, no detailed information on risk frequency and consequences is provided. Moreover, the systems can only be used in sites where a TV/VCR is available. Finally, all such systems do not allow for easy and accurate reproduction of the entire informed consent process.

Accordingly there is a need for an improved informed surgical consent system and method which (1) explains the entire surgical procedure in an easy-to-understand fashion, (2) is complete and consistent, (3) allows the patient unlimited time to review the information until a full understanding is achieved, (4) requires minimal time from the surgeon, (5) requires the patient to acknowledge in writing each potential surgical complication, (6) decreases surgeon liability, (7) is available for use 24 hours a day, 7 days a week at all patient care sites, and (8) which automatically and reliably tracks the outcomes of surgeries.

### SUMMARY OF THE INVENTION

A system for obtaining informed consent from a patient undergoing a surgery is disclosed. The system comprises a visual representation of the surgery, an auditory component integral with the visual representation, the audio component comprising a narration explaining the visual representation of the surgery, a textual component integral with the graphic representation and the auditory component, the textual component comprising a summary of each complication associated with the surgery, and an input mechanism for inputting an acknowledgment of each complication. The visual representation is preferably three-dimensional, and the textual component preferably includes a mechanism for flagging a complication about which the patient desires further information. The system further includes a storage device for electronically storing the patient's acknowledgment of each complication. The input means may further comprise a mechanism for identifying each complication which the patient encountered during the surgery, such that the system can determine an outcome of the surgery based on the identified complication(s). In a preferred embodiment, the system is web-based and available twenty-four hours a day.

Another embodiment of a system for obtaining informed consent from a patient undergoing a surgery is also disclosed. The system comprises a three-dimensional visual representation of the surgery, an auditory component integral with the visual representation, and a textual component integral with the visual representation and the auditory component, the textual component comprising a summary of each complication associated with the surgery. The textual component preferably includes a mechanism for identifying a complication about which the patient desires further information. The system further comprises an input mechanism for inputting an acknowledgment of each complication, and a storage mechanism for electronically storing the patient's acknowledgment of each complication. The input mechanism preferably includes a mechanism for identifying each complication that the patient encountered during the surgery, such that the system can determine the outcome of the surgery based on the identified complication(s). In a preferred embodiment, the system is web-based and available twenty-four hours a day.

A method of obtaining informed consent from a patient undergoing a surgery is also disclosed. The method comprises the steps of displaying the surgery visually to the patient, providing narration to accompany the visually displayed surgery, displaying a summary of each complication associated with the surgery, and requesting acknowledgment from the patient of each complication, wherein the visual representation, the narration, the summary, and the acknowledgment request are integrally combined in a single informed consent system. The surgery is preferably visually displayed in a three-dimensional form. The method may further comprise the step of requesting identification of each complication encountered by the patient during the surgery so that an outcome of the surgery can be determined based on the identified complication(s). The method may also further comprise the step of electronically storing the patient's acknowledgment of each complication.

Another embodiment of a method of obtaining informed consent from a patient undergoing a surgery is also disclosed. The method comprises the steps of displaying the surgery visually in a three-dimensional form to the patient, providing narration to the patient to accompany the visual representation, displaying a summary of each complication associated with the surgery, and requesting acknowledgment from the patient of each complication. The surgery is preferably displayed to the patient in a three dimensional form. The method may further comprise the step of requesting each complication about which the patient desires further information. The method may also further comprise the step of electronically storing the patient's acknowledgment of each complication.

While the principal advantages and features of the present invention have been explained above, a more complete understanding of the invention may be attained by referring to the description of the preferred embodiments.

### BRIEF DESCRIPTION OF THE FIGURES

FIG. 1 displays a schematic of one embodiment of an informed consent system in accordance with the present invention;

FIGS. 2A and 2B display exemplary graphic components of the informed consent system of FIG. 1;

FIG. 3 displays a flowchart of one embodiment of the informed consent system of FIG. 1 in operation;

FIG. 4 displays a flowchart of another embodiment of the informed consent system of FIG. 1 in operation; and

FIG. 5 displays an example of one complication displayed to a user of the informed consent system of FIG. 1.

### DETAILED DESCRIPTION OF THE PREFERRED EMBODIMENTS

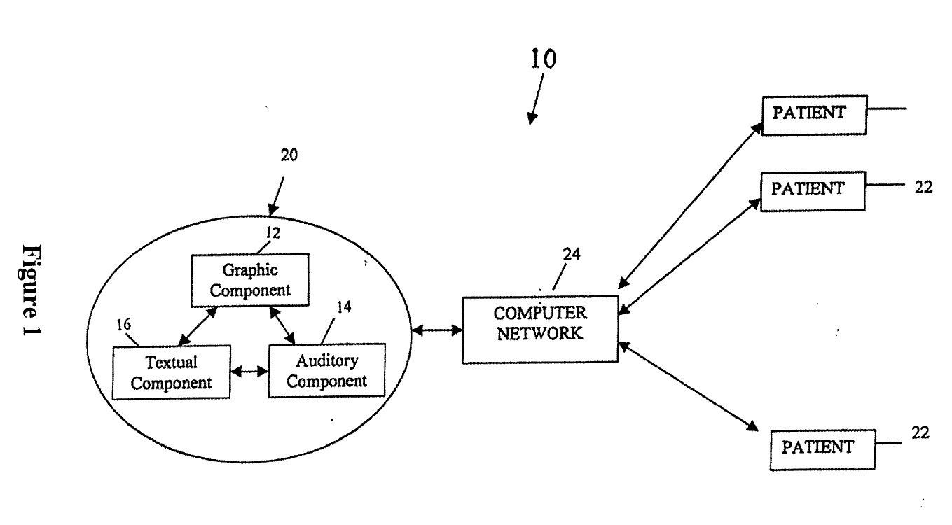

The present invention relates to an improved system and method of obtaining informed consent from a patient undergoing a surgery. Referring to FIG. 1, the system includes an informed consent system 20, preferably in the form of software, accessible to a plurality of patients 22 through a computer network 24, such as the Internet. In a preferred embodiment, the system 20 is made available to doctors and hospitals on a subscription basis through a web-based medium 24 hours per day, 7 days per week. A web-based system is ideal because it allows the surgeon access at all patient care sites. Surgeon offices are equipped with computers and Internet access, hospitals have computers with Internet access in every patient care area, and some hospitals even supply computers for use in patient rooms. Wireless Internet computers and hand held devices, also enhance patient accessibility. In addition, by automating the process, all of the information entered into the system 20 can be stored on a centralized and secure database for future use. It can be appreciated, however, that the system 20 can be a stand-alone system installed separately at each participating patient care site. In addition, while the system will be discussed with respect to a neurosurgical operation, namely an anterior cervical diskectomy, it can be appreciated that it can be used for any type of surgical operation.

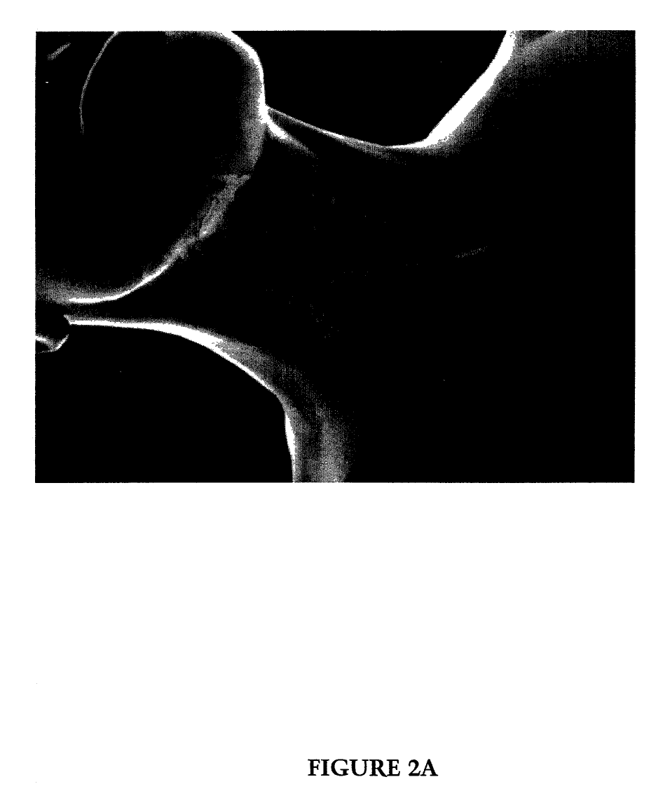

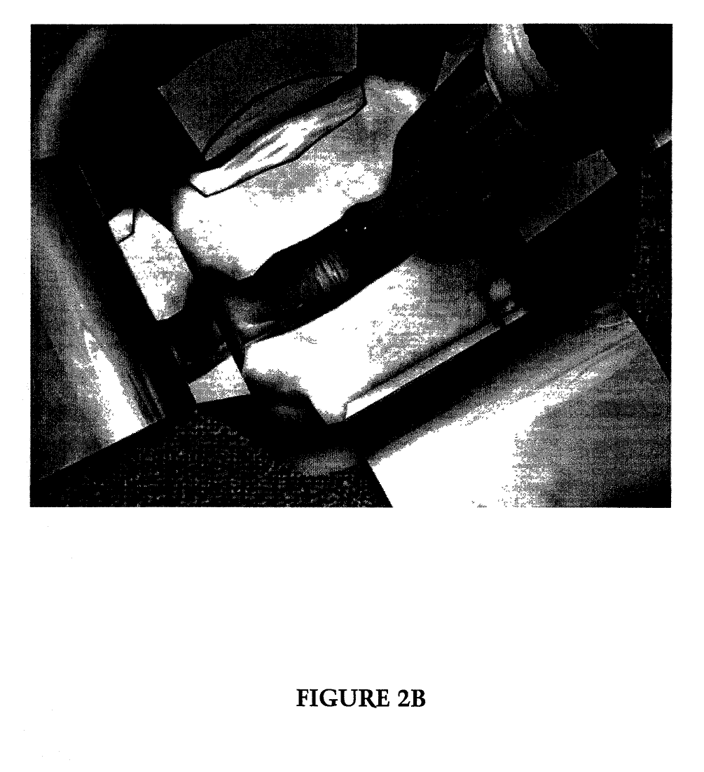

The informed consent system 20 includes a visual or graphic component 12, an auditory component 14, and a textual component 16 integral with the graphic component 12 and the auditory component 14. The visual component 12 consists of a detailed 3-dimensional (3D) animation of the surgery. Such animation permits all angles of the surgical anatomy and nearby important structures that could be damaged during surgery to be easily conveyed to the patient. The visual component 12 is preferably developed with input from experienced and respected surgeons in the pertinent field. In particular, the visual component 12 details each step of the surgery, from the start of the operation to its completion. One example of an animation is shown in a compact disc (CD) attached hereto as Appendix A and incorporated herein by reference. As shown, the animation demonstrates the tissues that are cut, removed, and/or repaired during surgery and their relation to other nearby important structures. Examples of two frames of such an animation are displayed in FIGS. 2A and 2B.

The auditory component 14 consists of narration accompanying the visual component 12 that explains the surgical anatomy, methods, and possible complications associated with each step of the procedure. The auditory component 14 for an Anterior Cervical Diskectomy and Fusion with Plating is included in the CD of Appendix A. The narrative script for this auditory component is displayed below. The “/////” within the script denotes the end of a step or stage of the surgery.

### EXAMPLE

### Narrative Script for Anterior Cervical Diskectomy and Fusion with Plating

1. Welcome to the Graphic Surgery Consent for an Anterior Cervical Diskectomy and Fusion with Plating.

2. Not every surgeon performs this operation exactly the same way, and every complication associated with this procedure cannot be predicted. However, the important steps used in almost all anterior cervical diskectomies, and most of the potential complications or problems associated with this surgery will be explained.

3. Your surgery is performed while under general anesthesia. Only after safely starting general anesthesia will the spine surgeon begin your surgery. If you wish more information about general anesthesia you should talk to your anesthesiologist. You can also ask if they offer the Graphic Surgery Consent for general anesthesia to their patients. //////////

4. Your surgeon will probably use special magnifying glasses or a microscope during at least part of this operation.

5. The first step in an anterior cervical diskectomy is for the surgeon to locate the area on your neck over the disk that is to be removed. The skin is cleaned with a combination of liquids such as hibicleanse, betadine, alcohol, iodine or another soap before starting the surgery. If you are allergic to any of these materials, you should tell your surgeon before surgery. The anesthesiologist will also give you an antibiotic through your IV before surgery starts, so you should be sure to inform both your surgeon and anesthesiologist of all your medication allergies. ///////////////

6. An incision about 1½ to 2 inches long is then made in or near one of the skin folds. If more than two disks are to be removed, a longer incision is used and may be in a different direction. Numbness in the area of the incision after surgery is not unusual.

7. After the skin has been opened, the muscle just below the skin called the platysma is divided. ///////////////

8. The surgeon then carefully develops a pathway between the muscles in the neck all the way down to the spine. This pathway can be developed using either surgical instruments or the surgeon's finger.

9. The pathway between the neck muscles is developed so that the esophagus, trachea, and nerve to the vocal cords are moved medially and the carotid artery and jugular vein are moved laterally. Before proceeding any further the surgeon usually checks to make certain that these structures are properly positioned.

10. Because the carotid artery, esophagus, trachea (commonly called the windpipe), and nerve controlling the vocal cords are so close to the area being operated on, there is a very small risk (usually less than 0.5% or 5 out of 1,000 people) that any or all of these structures could be damaged during the surgery.

11. If any of these structures are damaged, the surgery for removal of the disk may be stopped. Additional surgery is usually needed to repair damage to these structures, and another surgeon may be needed.

12. Hoarsenesss after this operation is very common and occurs in up to 15% of patients. The hoarseness is usually caused by swelling, is temporary, and improves over over several days. However, in 4% or less of patients the nerve controlling the vocal cords can be damaged, and the hoarseness may be permanent.

13. Special instruments called retractors are carefully placed in the wound to hold open this pathway, and keep the muscles, carotid artery, esophagus, and trachea out of the surgeon's way. ///////////////

14. Now the spine and disks can be seen. A needle is usually inserted into one of the disks, and an X-ray is taken to confirm the correct disk level. In patients who have thick necks from muscle or fat, the disks may be very difficult to see on X-ray, and in this situation there is a small risk of operating on the wrong disk.

15. When the surgeon is sure of the disk to be operated on, threaded pins are inserted into the bones above and below the disk and a special instrument is used to help open up the disk space. ///////////////

16. The disk is cut open and removed using a variety of grasping instruments and drills.

17. A ligament just behind the disk can also be opened after the disk has been removed to check for any additional pieces of herniated disk.

18. If there are enlarged pieces of bone (called bone spurs) pushing on either the spinal cord or the nerves, they are carefully removed with special instruments.

19. During removal of the disk and bone spurs there is a small risk that the spinal cord, nerves, or the covering around the spinal cord can be damaged.

20. Damage to the spinal cord or nerves during surgery is rare but can result in numbness, tingling, muscle weakness or even paralysis. Damage to the spinal cord occurs in 3% or less of patients and occurs most often in those patients who have signs of a spinal cord problem before surgery (usually called a myelopathy). Damage to individual nerves is even more rare and happens in only about 0.3% of patients.

21. In about 2% or less of patients the covering around the spinal cord and nerves is torn, allowing fluid to leak out. The tear can usually be directly repaired and causes no further problems. ///////////////

22. After the disk and bone spurs have been removed, the cartilage is scraped off the edges of the bones above and below the disk, so that the bones can grow together.

23. There is a risk that too much bone can be removed during surgery, and if this happens a fusion and plating may be needed even if not originally planned. ///////////////

24. If a fusion is performed, bone (either from a bone bank or from your hip) is carefully measured, shaped, and inserted into the empty disk space. There is a very small risk that the bone can be placed in too far and put pressure on the spinal cord.

25. After surgery, in up to 13% of cases the inserted bone comes out of the disk space. If the bone does come out it usually does not cause any problems, but in very rare cases it can put pressure on nearby important structures such as the esophagus or trachea.

26. The pins used to help separate the bones are removed. ///////////////

27. A strong metal plate (usually titanium) is placed over the bone graft, and screws are placed into the bones above and below to secure the plate. This metal plate acts as an internal neck brace, improves bone healing (called fusion), and basically removes the risk of the bone coming out after surgery.

28. In anywhere from 8%-20% of patients undergoing plating the screws or plate can come loose or break. In most of these cases the loose or broken instrument does not cause any serious problems, but a surgery is usually performed to remove the or loose implant. It is extremely rare, but has been reported that a loose or broken implant can press on a nearby structure (such as the esophagus or trachea) and cause problems with swallowing or breathing.

29. There is an even smaller risk that one of the screws can damage a nerve, the spinal cord or a nearby disk (no #'s). ///////////////

30. At this point the surgery is almost finished. The surgeon stops any visible bleeding, and then washes out the wound with an antibiotic solution.

31. The wound is closed by placing sutures or stitches (which will dissolve) in the platysma muscle and the skin. Glue or small strips of tape are usually placed over the incision, and a bandage is placed over the area.

32. There is a risk of bleeding after surgery. If a large blood clot forms it can cause pressure on the spinal cord (0.9%) or problems with breathing or swallowing (1.5%). These large blood clots only occur in 1.5% or less of patients, but if it does occur emergency surgery may be required to remove the blood clot.

33. After surgery anywhere from 0.3%-3% of all patients suffer from an infection at the surgical site and may require treatment with antibiotics, wound care, or even additional surgery. The risk of infection is usually highest in patients undergoing repeat surgery and those with diabetes.

34. Occasionally the incision does not heal properly after surgery. Most cases of poor wound healing are caused by infections and/or severe diabetes. ///////////////

35. If a fusion has been performed a neck brace may be placed around your neck in the operating room to help the bone heal. In about 5% of patients who undergo a single level fusion (and about 15% of those who have fusions across more than one disk level) the bones do not heal properly. Most patients with poor bone healing have no problems, but if a patient does develop problems, another surgery is usually necessary.

36. The anesthesiologist then allows the patient to wake up and removes the breathing tube.

37. After you are awake and breathing well, you will be taken to the recovery room where you will stay until more fully awake.

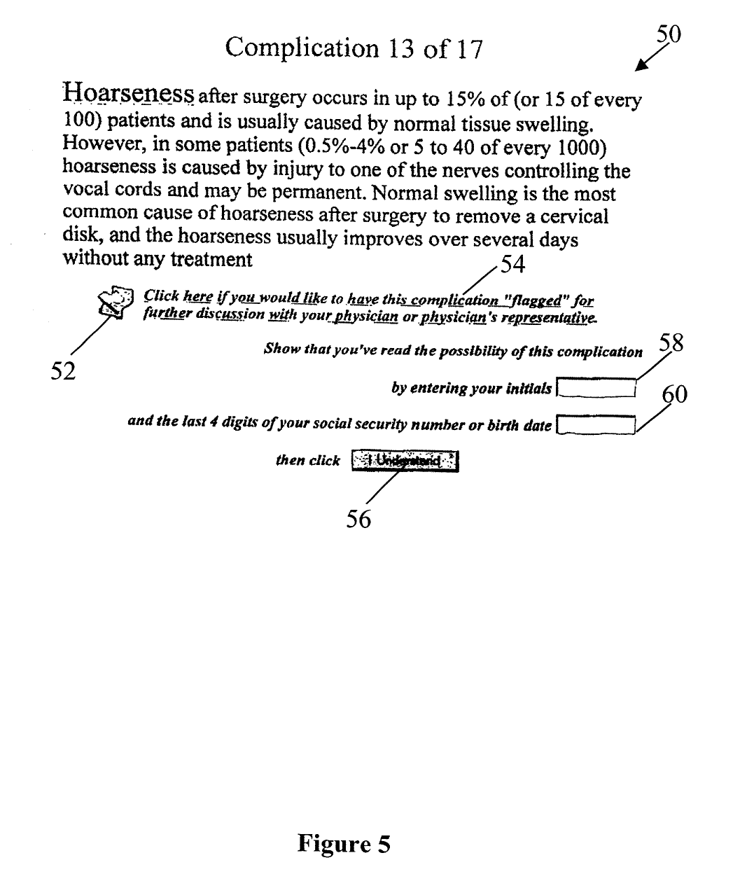

The textual component 16 consists of a textual report of the potential surgical complications, and further explains and reinforces the understanding of potential complications associated with the surgical procedure. Details relating to and explaining potential complications are based upon an exhaustive review of the scientific literature. The textual component 16 identifies each potential complication and requires the patient to acknowledge each complication in writing, preferably electronically. An example of a complication 50 displayed via the informed consent system 20 is shown in FIG. 5. Complication 50 includes a brief explanation of each complication and the frequency with which it occurs. If a patient desires further information about the complication, that complication can be flagged or identified for further review with the surgeon by clicking on the flag 52 or the hyperlink 54. The patient must acknowledge the complication by clicking on the acknowledge button 56. In a preferred embodiment, the patient must also input some personal information, such as his or her initials via input box 58 and the last four digits of his or her social security number or birth date via input box 60. The patient's acknowledgment is stored in a memory (not shown) of the informed consent system 20.

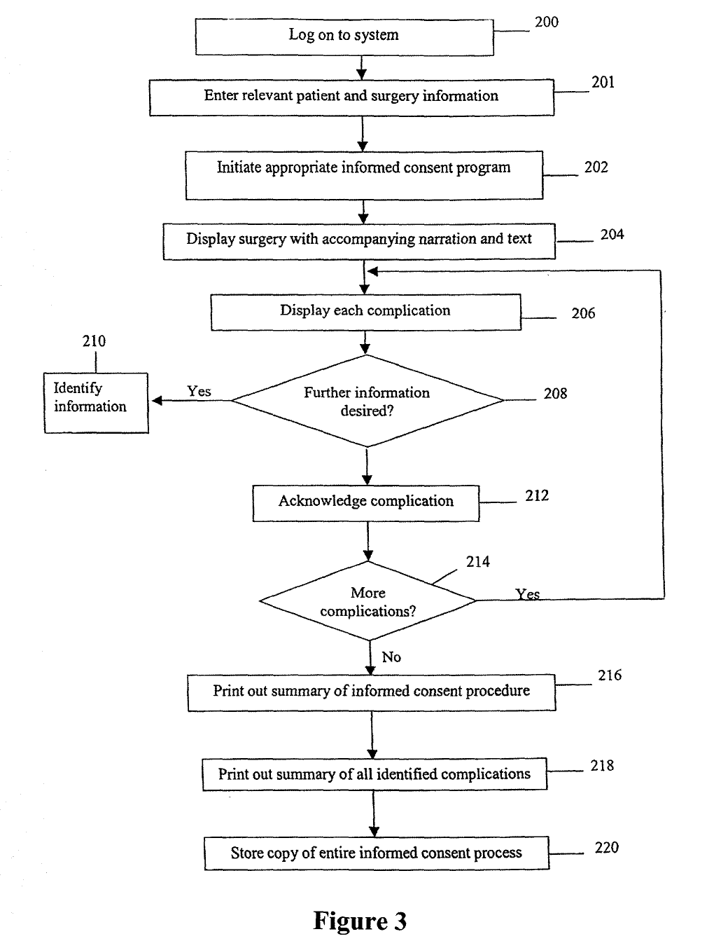

The informed consent system 20 will now be described in operation with reference to FIG. 3. At 200, a hospital staff person (i.e., secretary, nurse, doctor) enters a password to log on to the informed consent system 20. In a preferred embodiment, the password is associated with a particular surgeon. At 201, the hospital staff person enters relevant information about the patient (i.e., name, address, age, etc.), surgeon (if not tied with password) and the type of surgery. At 202, the patient initiates the appropriate informed consent program for his or her surgery. At 204, the surgery is visually displayed to the patient, along with the accompanying auditory and textual components. At 206, each of the potential complications associated with the surgery is then displayed in a textual format. At 208, a check is made whether the patient wishes to further discuss the complication. If so, at 210, the patient can identify the complication. At 212, the patient acknowledges the complication, which is stored by the informed consent system 20, preferably in a database form. In an alternative embodiment, all of the complications can be presented to the patient at one time. At 216, a summary of the informed consent is printed, and at 218, a summary of any identified complications is printed, all for inclusion in the physician and/or hospital record. At 220, a copy of the entire informed consent process is stored by the informed consent system 20, preferably in a centralized and secure database. In a preferred embodiment, the informed consent system 20 allows the patient to replay the entire procedure or segments thereof at any time.

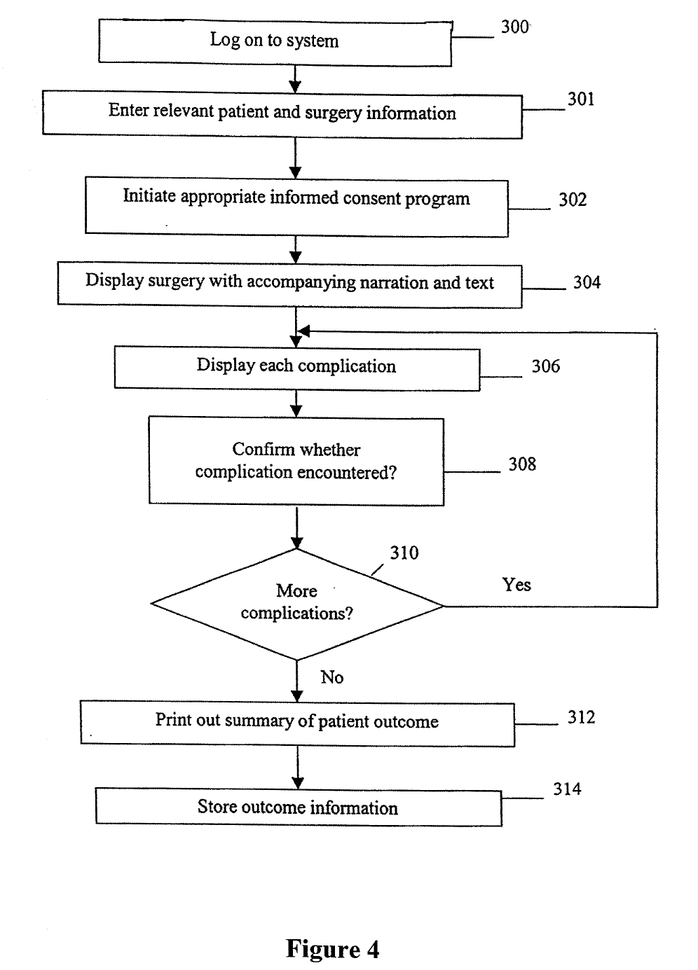

The informed consent system 20 may also be used post-surgery to track complications that actually occurred (i.e., the patient's outcome of the surgery). In particular, as shown in FIG. 4, at 300, a hospital staff person (i.e., secretary, nurse, doctor) enters a password to log on to the informed consent mechanism 20. At 301, the hospital staff person or the patient (i.e. user) enters relevant information about the patient and/or the surgery at issue to pull up the appropriate patient record. At 302, the appropriate informed consent program is initiated. At 304, the surgery is displayed with the accompanying auditory and text components. At 306, one of the complications associated with the surgery is displayed. At 308, the user is asked to confirm whether the patient encountered this complication. At 310, the system checks whether there are other complications associated with the surgery. If so, steps 306-310 are repeated until the user has gone through all of the complications associated with the surgery. Alternatively, all of the complications can be presented to the user at one time and the user can identify those that were encountered by the patient. At 312, a summary of the patient's outcome (i.e., all of the complications encountered by the patient) is printed out, and at 314, the outcome information is stored by the system.

By accumulating and storing such outcome data, complication rates for various surgical procedures can be determined. In addition, since complication history for each patient is tied to a particular surgeon, such rates can be broken down by surgeon. Such information is particularly useful for insurance companies setting premiums. For example, if a surgeon is well above or below the standard complication rate for a particular surgery, his or her premium can be adjusted accordingly.

Due to the nature of the informed consent system 20, it can be studied by the patient in the absence of a surgeon for an unlimited period of time until a satisfactory understanding of the material is achieved. As a result, the surgeon is allowed to perform other tasks and thus increase his or her efficiency. Furthermore, after the patient has completed review of the informed consent system 20, he or she is better equipped to discuss the surgery with the surgeon, thereby further reducing the amount of the surgeon's time required. Finally, since each of the components of the informed consent system 20 are integrally combined in a single mechanism, it can be easily and accurately reproduced for a deposition or in a court of law, thereby reducing the surgeon's or hospital's liability.

In a preferred embodiment, the informed consent system 20 is provided to surgeons and hospitals based on subscription rates. In the case of surgeons, the rates are based in turn upon the number of surgeons in a specialty and the malpractice rates for that specialty. In the case of hospitals, subscription rates are based in turn upon the number of specialty-related hospital beds. Based upon average neurosurgery practice values and the conservative assumption that the informed consent system 20 saves the surgeon ten (10) minutes for each informed consent, increased productivity can result in thousands of dollars savings per year per neurosurgeon, and a potential decrease in malpractice premiums.

The foregoing constitutes a description of various features of a preferred embodiment. Numerous changes to the preferred embodiment are possible without departing from the spirit and scope of the invention. Hence, the scope of the invention should be determined with reference not to the preferred embodiment, but to the following claims.

We claim:

1. A system for obtaining informed consent from a patient undergoing a surgery, comprising:
a visual representation of the surgery;
an auditory component integral with the visual representation, the audio component comprising a narration explaining the visual representation of the surgery;
a textual component integral with the graphic representation and the auditory component, the textual component comprising a summary of each complication associated with the surgery; and
input means for inputting an acknowledgment of each complication.

2. The system of claim 1, wherein the visual representation is three-dimensional.

3. The system of claim 1, wherein the textual component further comprises means for identifying a complication about which the patient desires further information.

4. The system of claim 1, wherein the input means further comprises means for identifying each complication which the patient encountered during the surgery, and wherein the system further comprises outcome determination means for determining an outcome of the surgery based on the identified complications.

5. The system of claim 1, wherein the system is web-based.

6. The system of claim 1, wherein the system is available twenty-four hours a day.

7. The system of claim 1, further comprising storage means for electronically storing the patient's acknowledgment of each complication.

8. A system for obtaining informed consent from a patient undergoing a surgery, comprising:
a three-dimensional visual representation of the surgery;
an auditory component integral with the visual representation;
a textual component integral with the visual representation and the auditory component, the textual component comprising a summary of each complication associated with the surgery.

9. The system of claim 8, wherein the textual component further comprises means for identifying a complication about which the patient desires further information.

10. The system of claim 8, further comprising input means for inputting an acknowledgment of each complication.

11. The system of claim 10, wherein the input means further comprises means for identifying each complication that the patient encountered during the surgery, and wherein the system further comprises outcome determination means for determining the outcome of the surgery based on the identified complication.

12. The system of claim 8, wherein the system is web-based.

13. The system of claim 8, wherein the system is available twenty-four hours a day.

14. The system of claim 8, further comprising storage means for electronically storing the patient's acknowledgment of each complication.

15. A method of obtaining informed consent from a patient undergoing a surgery, comprising:
displaying the surgery visually to the patient;
providing narration to accompany the visually displayed surgery;
displaying a summary of each complication associated with the surgery; and
requesting acknowledgment from the patient of each complication, wherein the visual representation, the narration, the summary, and the acknowledgment request are integrally combined in a single informed consent system.

16. The method of claim 15, wherein the surgery is visually displayed in a three-dimensional form.

17. The method of claim 15, further comprising the step of requesting identification of each complication encountered by the patient during the surgery so that an outcome of the surgery can be determined.

18. The method of claim 15, further comprising the step of requesting identification of each complication about which the patient desires further information.

19. The method of claim 15, further comprising the step of electronically storing the patient's acknowledgment of each complication.

20. A method of obtaining informed consent from a patient undergoing a surgery, comprising:
displaying the surgery visually in a three-dimensional form to the patient;
providing narration to the patient to accompany the visual representation;
displaying a summary of each complication associated with the surgery; and
requesting acknowledgement from the patient of each complication.

21. The method of claim 20, wherein the surgery is displayed to the patient in a three dimensional form.

22. The method of claim 20, further comprising requesting each complication about which the patient desires further information.

23. The method of claim 20, further comprising the step of electronically storing the patient's acknowledgment of each complication.
